(19) United States
(12) Patent Application Publication
(10) Pub. No.: US 2007/0255307 A1
(43) Pub. Date: Nov. 1, 2007

(54) Hemorrhoid Reduction Apparatus and Method

(76) Inventor: Alexander J. Cummings III, Peoria, IL (US)

Correspondence Address: HUSCH & EPPENBERGER, LLC, 190 CARONDELET PLAZA, SUITE 600, ST. LOUIS, MO, 63105-3441, US

(21) Appl. No.: 11/688,452

(22) Filed: Mar. 20, 2007

Related U.S. Application Data
(60) Provisional application No. 60/784,312, filed on Mar. 20, 2006.

(51) Int. Cl. A61M 19/00; A61F 13/20; A61M 31/00

(52) U.S. Cl. 606/197; 604/12; 604/514

(57) ABSTRACT

A hemorrhoid reduction device including a body having a longitudinal axis, an apex on the longitudinal axis and a bottom opposite the apex. The body has an outer side wall at an angle to the longitudinal axis such that the body generally narrows from the bottom to the apex. There is at least one ridge on the outer side wall, the at least one ridge being between the base and the apex, and the at least one ridge presents a biasing face substantially oriented towards the apex.

### CROSS-REFERENCE TO RELATED APPLICATIONS

This application claims priority from U.S. Provisional Patent Application No. 60/784,312 filed Mar. 20, 2006.

### BACKGROUND OF THE INVENTION

1. Field of the Invention

The field of the present invention is in personal health and hygiene equipment, in particular in equipment for ease of relief of hemorrhoid symptoms.

2. Related Art

Many people suffer from the pain and itch of swollen hemorrhoidal tissues. Hemorrhoids are characterized at least in part by abnormally dilated vasculature in the rectal tissue substantially adjacent to the anus. The condition becomes markedly symptomatic when this vasculature and the tissue in which it resides lose enough normal anatomical integrity to protrude outside the anus. That is, the tissue that, when healthy, remains internal and above the anal sphincter protrudes, when hemorrhoidal, outside the body, past the anal sphincter. Both the exposure of the mucosal tissues to the outside air and the constricting pressure of the anal sphincter on the protruding tissue cause the hemorrhoidal tissue to become acutely symptomatic, and uncomfortable.

Continuation of this protruded condition can cause the disease process to advance so that moderate cases become severe. Severe cases may require surgery. For pre-surgical mild to moderate cases, immediate comfort and the arrest of further deterioration of the tissue may be treated in a straightforward manner. The prolapsed or protruding tissue need merely be biased in an internal direction until it is replaced inside the anal sphincter near its pre-diseased normal anatomical position.

Replacing the affected tissue by pushing it is known in the prior art. In addition to simple digital placement, a device has been disclosed in the prior art in the form of a cylindrical device disclosed in U.S. Pat. No. 6,364,852 B1 to Lee.

The short comings of digital re-approximation and the Lee device are that the replacement modality is cylindrical. Cylindrical objects do not conform to the anatomical dynamics at hand. As a result, any advantages gained by replacement of the affected tissue with a cylindrical object are frequently lost upon removal of the cylindrical object. The anal sphincter is a ring of muscle which, like any other muscle, has in various patients greater or lesser degrees of resiliency and tone. Since retention of the affected tissue side the patient's rectum and above the level of the anal sphincter requires in most cases a gradual reclosure of the anal sphincter. Simple cylinders are not well configured to achieve this.

Moreover, simple cylinders have the shortcoming of not accommodating various possible dimensions of different patients and their affected tissue. Choosing an appropriate size device from a plurality of choices is problematic.

Additionally, in order to be effective, prior art solutions can be time consuming. For example, in the Lee reference it is stated that the use of the device may take up to one hour (see column 13 line 41).

Finally, prior art devices are impractical as a maintenance device as there is provided no component designed for disposable or one time use.

There is a need in the art for a single device that cannot only bias affected tissue into its normal anatomical position, but can facilitate retention of it there. There is further need in the art for a single device usable by different patients of varying anatomy. There is a further need in the art for a portable device that allows for disposable one time use while maintaining adequate sanitation. There is a continuing need in the art for devices that are comfortable and economical.

### SUMMARY OF THE INVENTION

It is in view of the above problems that the present invention was developed. The invention is a tapered hemorrhoid reduction device. The device has at least one biasing surface that is not parallel to an axis of reinsertion for the affected tissue. That is, in at least one embodiment, the device has a longitudinal axis and sidewalls that are tapered or frustoconical. The device may be further typified by at least one ridge substantially perpendicular to the longitudinal axis and disposed as a biasing face with which affected tissue may be re-inserted.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

The accompanying drawings, which are incorporated in and form a part of the specification, illustrate the embodiments of the present invention and together with the description, serve to explain the principles of the invention. In the drawings:

FIG. 1 is a cutaway side view of the reinsertion device of the present invention.

FIG. 2 is a top view of the of the reinsertion device.

FIG. 3 is a perspective view from the top of the reinsertion device.

FIG. 4 is a perspective view from the rear of the reinsertion device.

FIG. 5 is a perspective view of an alternative embodiment.

FIG. 6 is a side view of an alternative embodiment.

FIG. 7 is a perspective view of the device with a cover.

FIG. 8 is a side view of an alternative embodiment.

### DETAILED DESCRIPTION OF THE PREFERRED EMBODIMENTS

The following description of the preferred embodiment(s) is merely exemplary in nature and is in no way intended to limit the invention, its application, or uses.

Referring to the accompanying drawings in which like reference numbers indicate like elements, reduction device 10 is described in relation to a longitudinal axis 12. The longitudinal axis 12 be in any direction, provided that the device 10 is being used such that axis 12 is oriented substantially parallel with the patient's rectum, and therefore in line with a direction of bias for reinsertion of the affected tissue. The outer surface of the reduction device is characterized by an apex 14 and an outer side wall 16. The outer side wall 16, in the depicted embodiment, is generally frustoconical. Any reinsertion device with a sidewall that is tapered or angled relative to longitudinal axis is considered to be within the scope of the present invention. The angle may vary radially. The tapered side may be generally concave or convex towards the longitudinal axis, as shown at 216 and 316 in FIG. 8. Generally, the operative portion of the device is narrower towards the apex and wider as it progresses in a longitudinal direction away from the apex 14.

The depicted embodiment shows a further novel feature of the present invention, which is embodied by ridges 18 and 20. Any number of ridges may be included. Any dimension of ridge is considered to be within the scope of the present invention, although in preferred embodiments, the outer circumference of the ridge will graduate towards a progressively wider slanted or tapered outer face 16 as the sidewall becomes farther from the apex 14. Ridges 18 and 20 are characterized by presenting at least a partial face having a tangent that is sloped at a greater angle from the longitudinal axis than the side wall 16. In the depicted embodiment, the ridges actually curve, and that curve at its highest point 19 or 21 is characterized by a tangent that is perpendicular to the longitudinal axis 12. Any ridge, boss, projection, nubbin or other feature interrupting the smooth taper of the side wall 16 in a fashion to present a biasing face towards the affected tissue, is within the scope of the present invention. The ridges may be further dimensioned to define a recess 34A, 34B, where lubricant or medicant may be received for application during use.

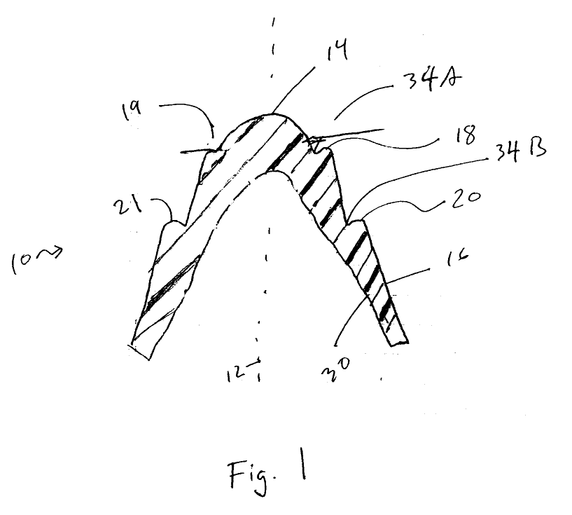

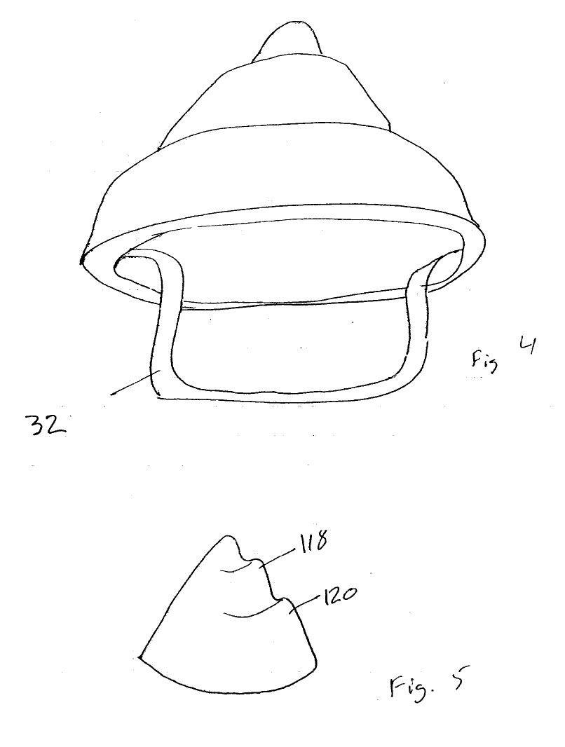

In the embodiment depicted in FIGS. 1-4, the reduction device 10 also has an inner face 30. This inner face may be concave as depicted, which conveniently affords a recess in which the users finger may be placed for responsive manipulation of the device. In alternative embodiments, such as that depicted in FIG. 4, a further feature may be included in order to ease retraction of the device such as handle 32. Any type of handle is considered to be within the scope of the present invention.

The present invention may be fabricated from any material, for example smooth hard plastic, smooth resiliently flexible plastic or rubber.

In operation, a user uses digital control of the device 10 by placement of a finger against inner wall 30. The apex 14 is aligned towards the affected tissue, and further towards the opening of the anal sphincter. The user simply pushes in the direction of the longitudinal axis 12, as properly oriented, bringing the apex 14 flush against the anal orifice. The side walls 16 contact that portion of the affected tissue closest to the actual anal orifice. Further pressure applied by the user causes the apex to advance internally, the affected tissue slides along the external surface 16 until it comes into contact with a first ridge 18. The ridge 18 provides a biasing surface against which the tissue may have the requisite pressure applied against it in order to bias it inwardly, towards and through the anal orifice. Where required, the affected tissue may continue to slide along the outer wall 16 until it comes in contact with a second ridge 20, and successive ridges (not depicted) until all of the affected tissue has been captured by the device and is receiving inwardly biasing pressure. Pressure is then continued until the affected tissue is biased past the anal sphincter, pushed internally and re-approximated with its normal anatomical position.

Upon gradual retraction of the reduction device 10, the anal sphincter is facilitated in maintaining pressure against the outer wall 16 of the tapered device. In so doing, a gradual reduction in size of the anal orifice while maintaining tension of the sphincter muscle serves to retain the affective tissue at its normal anatomical position throughout retraction of the device and thereafter.

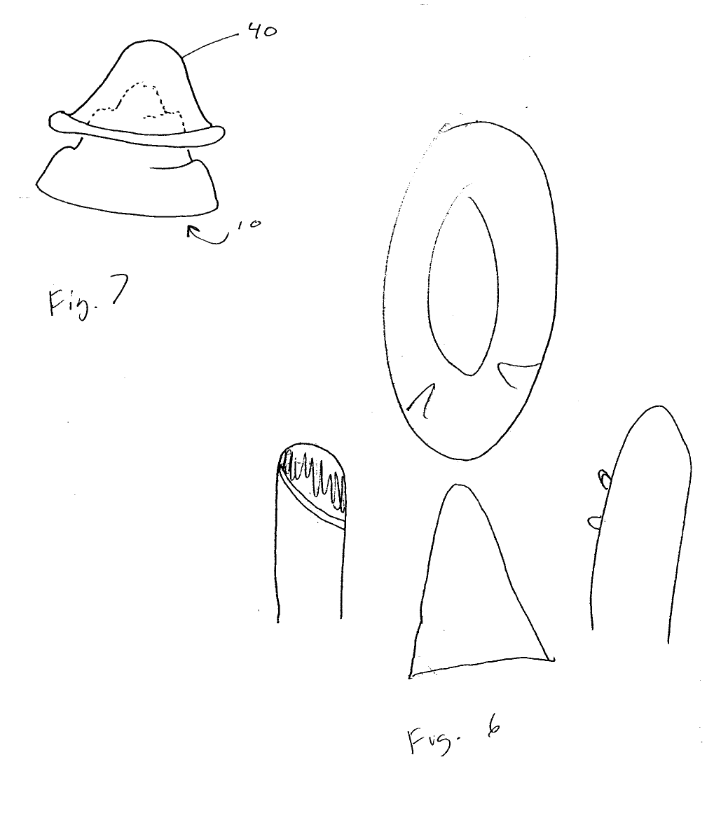

FIG. 5 depicts an alternative embodiment of the present invention. Because the anatomy of the affected tissue is seldom symmetrical, it may be advantageous in some circumstances that the ridges 118 and 120 also be asymmetrical. The ridges may be partially circumferential around the device to any degree. In a preferred embodiment, a partially circumferential embodiment would have ridges extending between 90 and 180 degrees around the longitudinal axis. An opposite side may be smooth and ridgeless.

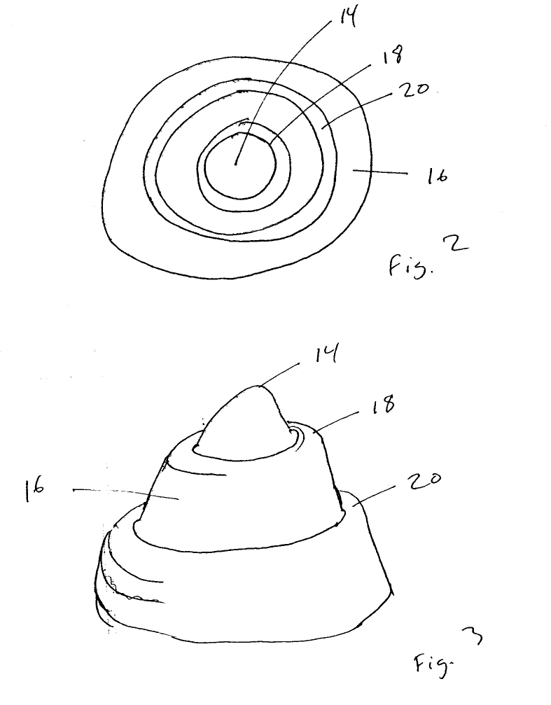

Another alternative embodiments, the device may not be circular as depicted in FIG. 2 when viewed from above, but may be oval shaped. Other shapes are considered to be within the scope of the present invention. The configuration of the ridges, moreover, need not be normal to the longitudinal axis 12. They may also be helical, spiral or otherwise. The ridges may be of different widths relative to each other.

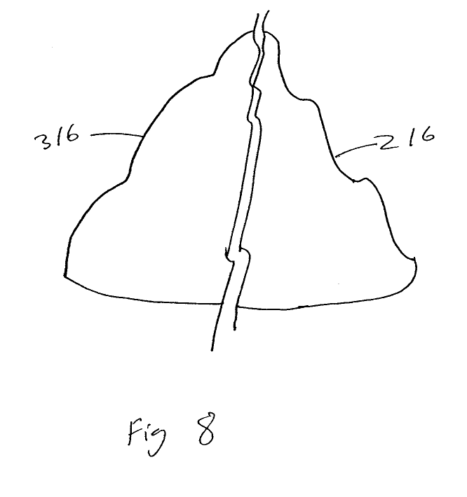

FIG. 7 depicts an optional, disposable device which may be employed with any alternative embodiment of the actual reduction device 10. The disposable cover 40 is a separate unit that may be removed from packaging, placed over the reduction device where it is dimensioned to be retained during use. The cover may be of any appropriate material, as for example paper or latex. In operation, the cover is placed by the user over the apex 14 and sidewall 16 of the reduction unit 10. The reduction unit is used as described above and retracted as described above. The cover may then be discarded and the reduction device 10 retained for further use. In this manner, maintaining sanitation of the device may be achieved after uses at locations where washing the reduction device 10 may be otherwise impracticable as for example in public laboratories.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A hemorrhoid reduction device comprising:
a body having a longitudinal axis, having an apex on said longitudinal axis and having a bottom opposite said apex;
said body having an outer side wall being at an angle to said longitudinal axis such that said body generally narrows from said bottom to said apex;
at least one ridge on said outer side wall, said at least one ridge being between said base and said apex; and
said at least one ridge presenting a biasing face substantially oriented towards said apex.

2. The device of claim 1 wherein said at least one ridge is partially circumferential.

3. The device of claim 1 wherein said biasing face is normal to said longitudinal axis.

4. The device of claim 1 wherein said at least one ridge is helical.

5. The device of claim 1 further comprising a disposable cover.

6. The device of claim 1 further comprising a concavity on said base of said body.

7. The device of claim 1 further comprising a handle disposed substantially on said base.

8. The device of claim 1 wherein an outer diameter of a first ridge is smaller than an inner diameter of a second ridge.

9. The device of claim 1 wherein said body is plastic.

10. The device of claim 1 wherein at least of one said ridges further comprises a recess.

11. The device of claim 10 wherein said recess is disposed and dimensioned to receive a lubricating ointment.

12. The device of claim 11 wherein said ointment is medicated.

13. The device of claim 1 wherein said base is circular.

14. The device of claim 1 where said base is oval.

15. The device of claim 1 wherein said outer sidewall is generally concave towards said longitudinal axis.

16. The device of claim 1 wherein said longitudinal axis is generally convex towards said longitudinal axis.

17. The device of claim 2 wherein said at least one ridge extends from about 90 degrees to about 180 degrees in circumference around said body.

18. The device of claim 1 further comprising at least one other ridge.

19. The device of claim 18 wherein said at least one ridge and said at least one other ridge are differently dimensioned.

20. The device of claim 19 wherein said different dimension is a different width.

21. The device of claim 19 wherein said different dimension is an angle.

22. The device of claim 19 wherein said different dimension is a curvature.

23. The device of claim 1 wherein said angle of outer side wall varies radially.

24. A method of making a hemorrhoid reduction device comprising:
establishing an apex along a longitudinal axis; tapering at least one section of outer side wall in relation to said longitudinal axis; and
fabricating at least one ridge on said outer side wall such that such at least one ridge presents a biasing face oriented toward said apex, said biasing face having an angle to said longitudinal axis greater than said angle of said outer side wall.
